(19) United States
(12) Patent Application Publication
(10) Pub. No.: US 2018/0369136 A1
(43) Pub. Date: Dec. 27, 2018

(54) DEVICES AND METHODS FOR TRANSDERMAL TREATMENT OF BASAL CELL CARCINOMA

(71) Applicant: NORTH CAROLINA STATE UNIVERSITY, Raleigh, NC (US)

(72) Inventors: Roger Narayan, Raleigh, NC (US); Russell Hall, Raleigh, NC (US); Yunyan Zhang, Raleigh, NC (US)

(21) Appl. No.: 16/065,221

(22) PCT Filed: Dec. 27, 2016
(86) PCT No.: PCT/US2016/068717

§ 371 Date: Jun. 22, 2018

Related U.S. Application Data
(60) Provisional application No. 62/271,697, filed on Dec. 28, 2015.

(51) Int. Cl. A61K 9/00; A61K 31/496; A61K 31/593; A61M 37/00

(52) U.S. Cl. CPC A61K 9/0021; A61K 31/496; A61K 31/593; A61M 2037/0061; A61M 2037/0023; A61M 2037/0046; A61M 37/0015

(57) ABSTRACT

Various implementations include a transdermal drug delivery system for treating basal cell carcinoma. The system includes a pharmacologic agent and an array of microneedles for creating pores in the skin surface and the stratum corneum layer of the epidermis, thereby enabling transdermal delivery of the pharmacologic agent. The pharmacologic agent includes itraconazole and/or vitamin D3. Various implementations also include a method of treating basal cell carcinoma in a subject. The method includes: (1) applying an array of microneedles to a skin site having basal cell carcinoma, such that the microneedles penetrate the surface of the skin and the stratum corneum layer of the skin; and (2) administering a pharmaceutical composition to the subject intradermally through openings in the skin formed by the microneedles, wherein the pharmaceutical composition comprises a therapeutically effective amount of a pharmacologic chosen from itraconazole, vitamin D3, or a combination thereof.

### CROSS REFERENCE TO RELATED APPLICATIONS

This application claims priority to U.S. Application No. 62/271,697, filed Dec. 28, 2015, and entitled “Devices and Methods for Transdermal Treatment of Basal Cell Carcinoma,” the content of which is herein incorporated by reference in its entirety.

### BACKGROUND

Basal cell carcinoma is a condition in which the cells in the deepest layer of the epidermis exhibit uncontrolled proliferation. This condition is believed to occur due to many genes, with each gene providing relatively weak individual contribution. Basal cell carcinoma lesions are appear as open sores, pink growths, red patches, shiny bumps, and/or scars. 2.8 million individuals were diagnosed with basal cell carcinoma in the United States in 2010, and 3,000 deaths per year are attributed to basal cell carcinoma. This condition is the most common cancer among Caucasians and Hispanics. The risk of developing basal cell carcinoma is associated with (a) exposure to midrange ultraviolet B radiation, (b) family history of melanoma, (c) red/blond hair, (d) indoor tanning, (e) the presence of a higher number of extremity moles, (f) a higher susceptibility to sunburn as a child/adolescent, and (g) a higher lifetime number of severe sunburns.

As noted in the National Comprehensive Cancer Network (NCCN) Clinical Practice Guidelines in Oncology: Basal Cell and Squamous Cell Skin Cancers, treatment of basal cell carcinoma is based on tumor depth, size, and location. The goals of treatment are elimination of cancerous tissue, maximal preservation of physical appearance, and maximal preservation of function. Surgery is commonly used to treat basal cell carcinoma. For example Mohs micrographic surgery is an effective technique. However, surgery may result in significant morbidity and may result in disfiguring scarring. In addition, basal cell carcinoma near the brain or eyes may be difficult to surgically treat without significant morbidity or scarring.

Due to the relatively benign nature of basal cell carcinoma, non-surgical treatment options, including pharmacologic treatment options, have been developed. For example, 5-fluorouracil, an antineoplastic agent, was the first pharmacologic agent to be approved by the U.S. Food and Drug Administration for topical treatment of superficial basal cell carcinoma. However, use of this agent is associated with side effects such as allergic reactions, dyspigmentation, inflammation, pain, and erosion. In addition, in vitro studies involving yeast and bacteria cells as well as in vivo studies involving immunosuppressed syngeneic mice have shown that fluorouracil causes mutations. 5% imiquimod, a synthetic immune modulator, has also been approved by the US Food and Drug Administration for topical treatment of superficial basal cell carcinoma. It should be noted that topical use of imiquimod may be associated with intense local inflammatory reactions, including skin weeping, erosion, and scabbing.

Itraconazole is a triazole that has been approved since 1992 for use in treatment of a wide variety of fungal infections, including onychomycosis (nail infection), aspergillosis, blastomycosis, candidiasis, crypotococcosis, histoplasmosis, and sporotrichosis. Inhibiting cell membrane function, interfering with cytochrome P450 activity, and reducing in ergosterol synthesis provides itraconazole with its antifungal activity. In 2010, a second mechanism of action was proposed for itraconazole, which involved suppression of the critical Hedgehog signaling pathway activity (Smoothened). The antineoplastic activity of itraconazole was studied using an in vivo murine model and showed that itraconazole suppressed growth of medulloblastoma (brain tumor) at serum levels comparable to those used in antifungal treatments. In addition, adverse events seen with other drugs that act via Hedgehog pathway inhibition are apparently absent with itraconazole. Subsequently, itraconazole was noted as showing Hedgehog signaling pathway suppression activity in the presence of all resistance-conferring Smoothened mutations. And, in 2012, a 30% decrease in basal cell carcinoma tumor diameter and improved wound healing among patients who received oral itraconazole was demonstrated. More recently, a clinical study was performed in which patients with a basal cell carcinoma tumor were treated with oral itraconazole. They showed that itraconazole reduced tumor area by 24% and reduced cell proliferation by 45%. It is important to note that use of oral itraconazole was associated with side effects, including grade 4 congestive heart failure. In addition to congestive heart failure, liver failure is a side effect of oral itraconazole therapy. In some cases, liver failure occurred in patients who had no underlying medical condition and no pre-existing liver condition.

In addition, vitamin D3 has been shown to suppress Hedgehog signaling in basal cell carcinoma in an in vitro model. However, vitamin D3 is a fat soluble vitamin and does not readily dissolve in aqueous media.

Thus, there is a need in the art for devices and methods for more effectively treating and preventing skin cancer.

### BRIEF SUMMARY

Various implementations include a transdermal drug delivery system for treating basal cell carcinoma. The system includes a pharmacologic agent and an array of microneedles for creating pores in the skin surface and the stratum corneum layer of the epidermis, thereby enabling transdermal delivery of the pharmacologic agent. The pharmacologic agent includes itraconazole and/or vitamin D3.

In some implementations, each microneedle has a diameter of less than 300 micrometers and a length of between 50 and 900 micrometers (e.g., 800 micrometers). In some implementations, the microneedles are lancet shaped.

In some implementations, the microneedles are biodegradable. In some implementations, the microneedles are polyglycolic acid (PGA) polymer microneedles. And, in some implementations, the microneedles are metal.

In some implementations, the pharmacologic agent is disposed onto the surfaces of the microneedles via piezoelectric inkjet printing.

In some implementations, the distal ends of the microneedles deliver the pharmacologic agent to a skin site having basal cell carcinoma below a stratum corneum layer of the skin site.

In some implementations, the array of microneedles include a circular array of microneedles. For example, in one implementation, the array of microneedles occupies an area of 1 square centimeter.

In some implementations, the array of microneedles includes a rectangular array of microneedles.

Various implementations include a method of treating basal cell carcinoma in a subject. The method includes: (1) applying an array of microneedles to a skin site having basal cell carcinoma, such that the microneedles penetrate the surface of the skin and the stratum corneum layer of the skin; and (2) administering a pharmaceutical composition to the subject intradermally through openings in the skin formed by the microneedles, wherein the pharmaceutical composition comprises a therapeutically effective amount of a pharmacologic chosen from itraconazole, vitamin D3, or a combination thereof.

In some implementations, the pharmaceutical composition is applied intradermally after removing the microneedles from the skin.

In some implementations, the microneedles are hollow and define an opening at the distal end of each microneedle, and the pharmaceutical composition is applied intradermally through the openings of the microneedles.

In some implementations, the surfaces of the microneedles penetrate the surface of the skin and the stratum corneum layer of the skin. And, the pharmacologic composition is administered to the skin site having basal cell carcinoma until the basal cell carcinoma is no longer present, according to a further implementation.

### BRIEF DESCRIPTION OF THE DRAWINGS

Various implementations of the device are explained in detail in the following exemplary drawings. The drawings are merely exemplary to illustrate the structure of the devices and certain features that may be used singularly or in combination with other features. The invention should not be limited to the implementations shown.

FIG. 1 illustrates a top schematic view of a transdermal drug delivery device according to one implementation.

FIG. 2 illustrates a partial perspective view of a transdermal drug delivery device according to another implementation.

FIG. 3 illustrates a side view of a transdermal drug delivery device according to another implementation.

FIGS. 4A-4C illustrate staining of skin grafts after three different treatment methods.

### DETAILED DESCRIPTION

Various implementations of the invention include systems and methods of topically delivering itraconazole and/or vitamin D3 to skin sites having basal cell carcinoma.

According to various implementations, a device having an array of microneedles is used to deliver itraconazole and/or vitamin D3 to the tumor site. The microneedles are used to produce conduits in the keratinized stratum corneum layer of the epidermis, which is about 15 micrometers thick and normally hinders transport of pharmacologic agents through the skin. Due to the small dimensions of the microneedles, which may be less than 300 micrometers in diameter, bleeding and other tissue damage at the injection site are minimized. Microneedles used for transdermal drug delivery are associated with low levels of patient pain since these devices do not enter deeper layers of the skin, where Meissner's corpuscles, Pacinian corpuscles, and large nerve endings are located. Furthermore, the microneedles provide a suitable mechanism for the topical delivery of itraconazole because itraconazole exhibits poor solubility in ethanol and water, making it not suitable for application to the skin surface using a liquid solution or a patch.

Various implementations of topically delivering itraconazole and/or vitamin D3 provides several advantages over systemic delivery of these pharmacologic agents, including the ability to deliver a high concentration of the pharmacologic agents to the tumor site while minimizing or diminishing systemic absorption, side effects, and toxicity. In addition, topical delivery of itraconazole and/or vitamin D3 improves wound healing and reduces basal cell carcinoma tumor diameter. Furthermore, customized therapies based on knowledge of basal cell carcinoma lesion geometry may be more effective than therapies based on a “one size fits all” philosophy.

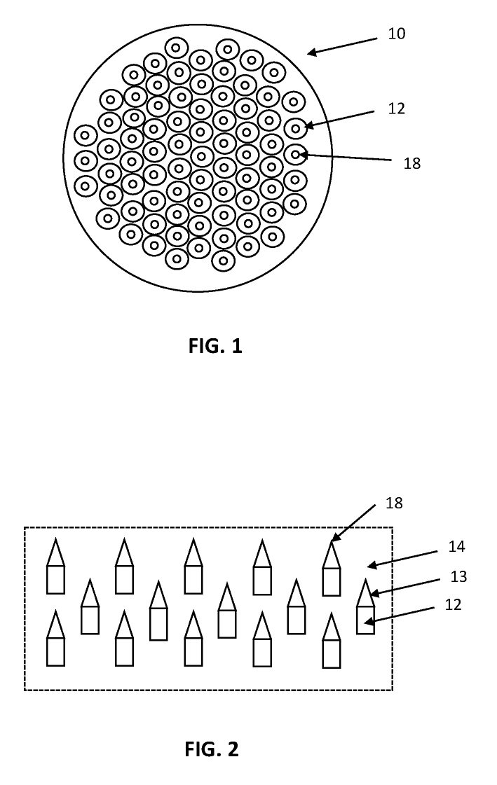

FIG. 1 illustrates an exemplary transdermal drug delivery device 10 according to one implementation. The device 10 includes a circular array of microneedles 12 disposed on a substrate 14. For example, this circular array may occupy 1 square centimeter. Each microneedle 12 in the array has a diameter of less than 300 micrometers and a length of between 50 and 900 micrometers (e.g., 800 micrometers). A pharmaceutical composition comprising a therapeutically effective amount of a pharmacologic agent (e.g., a pharmacologic agent chosen from itraconazole, vitamin D3, or a combination thereof) may be applied intradermally through the microneedles 12.

In another implementation, such as shown in FIG. 2, the pharmaceutical composition comprising a therapeutically effective amount of a pharmacologic agent (e.g., a pharmacologic agent chosen from itraconazole, vitamin D3, or a combination thereof) may be disposed on at least a portion of an external surface 13 of the microneedles 12 and the substrate 14. Furthermore, in yet another implementation, the microneedle device may be applied to a patient's skin to form conduits in the keratinized stratum corneum layer of the epidermis, and the pharmaceutical composition comprising a therapeutically effective amount of a pharmacologic agent (e.g., a pharmacologic agent chosen from itraconazole, vitamin D3, or a combination thereof) may be applied intradermally through these conduits by topical pipetting or other suitable method after the microneedle device is removed from the skin.

In some implementations, the microneedles 12 include lancet shaped distal ends 18. However, in other implementations, the distal ends 18 of the needles 12 may be any suitable shape for hypodermic use.

The microneedles 12 may be made from metal (e.g., stainless steel or other suitable metal) or from a suitable biodegradable polymer, such as polyglycolic acid (PGA), for example.

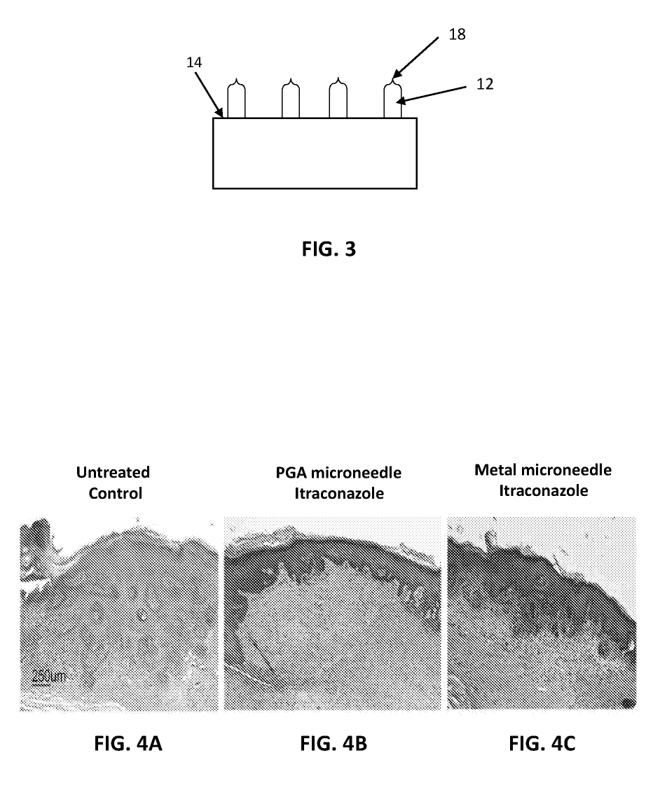

In other implementations, the microneedles 12 may be arranged in a rectangular array, such as the 1×4 array shown in FIG. 3.

Pharmaceutical compositions can comprise a therapeutically effective amount of a pharmacologic agent chosen from itraconazole, vitamin D3, or a combination thereof. For example, in some cases, the pharmaceutical composition can comprise a therapeutically effective amount of one of itraconazole and vitamin D3 that is administered until resolution of the basal cell carcinoma. In some cases, the pharmaceutical composition can comprise a therapeutically effective amount of itraconazole and vitamin D3. In some implementations, the treatment may include applying itraconazole and vitamin D3 alternately (or together). The dosage of itraconazole and/or vitamin D3 may depend on the thickness and mass of the basal cell carcinoma lesion, according to some implementations. And, in other implementations, the treatment may include applying one of itraconazole or vitamin D3 until resolution of the tumor.

The term “therapeutically effective amount” as used herein, refers to a dosage effective to alleviate or inhibit progress of cancer. In the case of methods of treating basal cell carcinoma, a therapeutically effective amount of a pharmacologic agent can be an amount effective to reduce the growth rate of basal cell carcinoma, halt the growth rate of basal cell carcinoma, or induce the death of basal cell carcinoma. For example, in some implementations, itraconazole doses may be between 12.5 mg/kg and 25 mg/kg.

The amount of pharmacologic agent (e.g., itraconazole, vitamin D3, or a combination thereof) present in the pharmaceutical composition can be an amount sufficient to cause a therapeutic effect but is low enough not to cause substantial intolerable adverse side effects. As used herein, “substantial intolerable adverse side effects” include those effects caused by either the delivery system or the active pharmaceutical agent which are incompatible with the health of the user or which are so unpleasant as to discourage the continued use of the composition. In some cases, the pharmaceutical composition can comprise 0.1 to 90 weight percent (e.g., 1 to 50 weight percent, or 1 to 30 weight percent) of a pharmacologic agent (e.g., itraconazole, vitamin D3, or a combination thereof), based on the total weight of the composition.

Pharmaceutical compositions can, if desired, include one or more pharmaceutically acceptable excipients. The term “excipient” herein means any substance, not itself a pharmacologic agent, used in conjunction with the pharmacologic agent delivered to a subject or added to a pharmaceutical composition to improve one of more characteristics, such as its drug delivery, handling, or storage properties, or to permit or facilitate formation of a dose unit of the composition. Excipients include, by way of illustration and not limitation, solvents, thickening agents, penetration enhancers, wetting agents, lubricants, emollients, substances added to mask or counteract a disagreeable odor, fragrances, and substances added to improve appearance or texture of the composition or drug delivery system. The foregoing list of excipients is not meant to be exhaustive but merely illustrative as a person of ordinary skill in the art would recognize that additional excipients could be utilized.

Compositions can be prepared by any technique known to a person of ordinary skill in the art of pharmacy, pharmaceutics, drug delivery, pharmacokinetics, medicine or other related discipline. For example, compositions can be prepared by admixing one or more excipients with a pharmacologic agent to form the pharmaceutical composition.

In some embodiments, the composition can comprise a penetration enhancing agent. Non-limiting examples of penetration enhancing agents include sulfoxides such as dimethylsulfoxide and decylmethylsulfoxide; surfactants such as sodium laurate, sodium lauryl sulfate, cetyltrimethylammonium bromide, benzalkonium chloride, poloxamer (231, 182, 184), tween (20, 40, 60, 80) and lecithin; the 1-substituted azacycloheptan-2-ones, particularly 1-n-dodecylcyclazacycloheptan-2-one; fatty alcohols such as lauryl alcohol, myristyl alcohol, oleyl alcohol and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate, amides and other nitrogenous compounds such as urea, dimethylacetamide (DMA), dimethylformamide (DMF), 2-pyrrolidone, l-methyl-2-pyrrolidone, ethanolamine, diethanolamine and triethanolamine, terpenes; alkanones, and organic acids, particularly salicylic acid and salicylates, citric acid and succinic acid. The permeation enhancer can be present from 0.1 to 30% weight percent depending on the type of permeation enhancer, based on the total weight of the composition.

The composition can comprise an antioxidant such as, for example, tocopherol or derivatives thereof, ascorbic acid or derivatives thereof, butylated hydroxyanisole, butylated hydroxytoluene, fumaric acid, malic acid, propyl gallate, or sodium metabisulfite or derivatives thereof. The antioxidant can be present in the composition in an amount of from 0.01 to 5 weight percent (e.g., 0.1 to 0.5 weight percent), depending on the type of antioxidant used, based on the total weight of the composition.

The composition can comprise a preservatives such as, but not limited to, benzalkonium chloride or derivatives thereof, benzoic acid, benzyl alcohol or derivatives thereof, bronopol, parabens, centrimide, chlorhexidine, cresol or derivatives thereof, imidurea, phenol, phenoxyethanol, phenylethyl alcohol, phenylmercuric salts, thimerosal, or sorbic acid or derivatives thereof. The preservative can be present from 0.01 to 10 weight percent, based on the total weight of the composition.

The composition can comprise one or more emulsifying agents. The term “emulsifying agent” refers to an agent capable of lowering surface tension between a non-polar and polar phase and includes compounds defined as “self-emulsifying” agents. Suitable emulsifying agents can come from any class of pharmaceutically acceptable emulsifying agents including, but not limited to, carbohydrates, proteins, high molecular weight alcohols, wetting agents, waxes and finely divided solids. The emulsifying agent can be present in the composition in a total amount of 1 to 15 weight percent (e.g., from 0.5 to 5 weight percent), based on the total weight of the composition.

The composition can comprise a pharmaceutical carrier or vehicle, such as one or more solvents. Examples of suitable carriers include C2-C10 alcohols, such as hexanol, cyclohexanol, benzyl alcohol, 1,2-butanediol, glycerol, and amyl alcohol; C5 -C10 hydrocarbons such as n-hexane, cyclohexane, and ethylbenzene; C4-C10 aldehydes and ketones, such as heptylaldehyde, cyclohexanone, and benzylaldehyde; C4-C10 esters, such as amyl acetate and benzyl propionate; ethereal oils, such as oil of eucalyptus, oil of rue, cumin oil, limonene, thymol, and 1-pinene; halogenated hydrocarbons having 2-8 carbon atoms, such as 1-chlorohexane, 1-bromohexane, and chlorocyclohexane. In some cases, the composition can comprise a water immiscible solvent, such as propylene glycol. In some cases, the pharmaceutical carrier can comprise an oil. Examples of oils comprise fats and oils such as olive oil and hydrogenated oils; waxes such as beeswax and lanolin; hydrocarbons such as liquid paraffin, ceresin, and squalane; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate.

The composition can comprise a thickening agent. Non-limiting examples of thickening agents include, include hydroxyalkylcelluloses and carboxyalkylcelluloses, such as, hydroxypropylcellulose (HPC), hydroxypropylmethylcellulose (HPMC), hydroxyethylcellulose (HEC), hydroxypropylethylcellulose (HPEC), methyl cellulose (MC), ethyl cellulose (EC), cellulose acetate (CA), cellulose acetate butyrate, cellulose acetate propionate, hydroxypropylmethylcellulose phthalate (HPMCP) (which is also an anionic polymer), carboxyl methylcellulose (CMC), cellulose acetate phthalate (CAP) (which is also an anionic polymer). Examples of pharmaceutically acceptable biologically derived materials include, but are not limited to, polysaccharides or their derivatives, such as, but not limited to, gums (such as, xantham gum, locust bean gum), sodium alginate, shellac, zein, and the like. It might also include anionic polymers such as polyacrylic acid, carboxypolymethylene, carboxymethylcellulose and the like, including derivatives of Carbopol polymers. Thickening agents can be present in an amount sufficient to provide the desired rheological properties of the composition.

Inventors have experimented with depositing pharmacologic agents with poor solubility in water and other polar solvents onto the surfaces of microneedles using piezoelectric inkjet printing. For example, an imidazole antifungal agent known as miconazole has been deposited onto the surface of GANTREZ AN 169 (benzene-free) microneedle arrays using piezoelectric inkjet printing. GANTREZ AN 119, 139, or 149 may also be used. The miconazole-coated microneedles underwent biodegradation on exposure to a moist jelly-like material (Sabouraud dextrose agar) and showed antifungal activity against the organism Candida albicans (ATCC 90028). This proof-of-concept study demonstrated that piezoelectric inkjet printing is a rapid, scalable, and low cost approach for loading pharmacologic agents, particularly pharmacologic agents with poor solubility in water, onto the surfaces of microneedles.

In implementations in which the pharmacologic agent is deposited on the surface of the device, piezoelectric inkjet printing may be used to deposit the pharmacologic agent onto the surfaces of microneedles 12. The microneedles 12 may then be used for local and transdermal treatment of basal cell carcinoma. However, in other implementations, the pharmacologic agent may be delivered intradermally using other suitable mechanisms.

To treat a skin site having basal cell carcinoma using the device 10, the device 10 is applied to the skin site, and the distal ends 18 of the microneedles 12 are urged below a stratum corneum layer of the skin site. The pharmacologic agent is delivered intradermally through the conduits formed in the skin by the microneedles 12.

FIGS. 4A through 4C illustrate the results from a study of the efficacy of using implementations of the above described device to treat basal cell carcinomas. In the study, keratinocytes isolated from foreskin samples were transduced with retrovirus encoding human SHH and seeded onto devitalized split-thickness human dermis prepared from abdominoplasty skin tissues. A one cm² section of mouse skin was removed down to fascia from the dorsum of each mouse and the skin composite was sutured to the mouse skin and dressed with polymoxin B antibiotic ointment, adaptic, telfa pad, and Coban wrap. The bandages were removed around two weeks after the surgery.

The animals were divided into three groups and treated daily with 50 μl 5 mg/ml itraconazole dissolved in 60% DMSO and 40% polyethylene glycol-400 solution for two weeks starting at two weeks after the surgery. The skin grafts of the animals in Group I were directly treated with topical pipetting of itraconazole solution. The skin grafts of the animals in Group II were poked 6 to 8 times spaced at 1 to 2 mm distance with an array of solid 4×4 solid polyglycolic acid (PGA) polymer microneedles followed by topical pipetting of itraconazole. The PGA polymer microneedles were made using injection molding and drawing lithography, and four 1×4 arrays were arranged in a parallel manner to create the 4×4 array. The skin grafts of the animals in Group III were injected with 50 μl itraconazole through a 1 ml syringe attached to a circular array of eighty-five hollow 800 micrometer tall 316L stainless steel microneedles arranged in 1 cm² area. The skin grafts were collected at the end-point and analyzed by hematoxylin and eosin (H&E) staining.

The PGA-microneedles used with the animals in Group II induced skin injuries in all of the animals as evidenced by traces of blood on the skin surface following each treatment. At the end of the 2-week treatment, only one of the two remaining PGA-treated animals had an apparent human skin graft. The wounding response was not apparent following injection with metal microneedle injections used with the animals of Group III. Two of the 4 mice in Group III treated with metal microneedles contained human skin grafts at the end-point.

For the animals in Group I, the epidermis of SHH-expressing control grafts displayed basaloid overgrowth that resembles human BCC as shown by H&E staining, which is shown in FIG. 4A. In contrast, the epidermal of the animals in Group II treated with PGA-microneedle mediated topical itraconazole were markedly thinner than that of the control graft, as shown in FIG. 4B. In addition, there was an increased presence of likely immune cells in the dermal compartment of the PGA-treated tissues. The epidermal tissues of the skin grafts of the animals in Group III were also thinner than the untreated tissues of Group I, as shown in FIG. 4C. These preliminary results indicate that microneedles may be used to facilitate delivery of itraconazole to treat basal cell carcinoma.

According to various implementations, the above described technology may be useful in treating basal cell carcinoma, imparting antineoplatic functionality to medical implants, and locally treating fungal infections (e.g., nail infections). In addition, applying one or more of these pharmacologic agents using microneedles can be used for prophylaxis and/or treatment of precancerous lesions since the microneedles are painless and are associated with minimal tissue damage and/or scarring.

The corresponding structures, materials, acts, and equivalents of all means or step plus function elements in the claims below are intended to include any structure, material, or act for performing the function in combination with other claimed elements as specifically claimed. The descriptions of various implementations of the present invention have been presented for purposes of illustration and description, but are not intended to be exhaustive or limited to the invention in the form disclosed. Many modifications and variations will be apparent to those of ordinary skill in the art without departing from the scope and spirit of the invention. The implementations were chosen and described in order to best explain the principles of the invention and the practical application, and to enable others of ordinary skill in the art to understand the invention for various implementations with various modifications as are suited to the particular use contemplated.

1. A transdermal drug delivery system for treating basal cell carcinoma, the system comprising a pharmacologic agent and an array of microneedles for creating pores in the skin surface and the stratum corneum layer of the epidermis thereby enabling transdermal delivery of the pharmacologic agent, the pharmacologic agent comprising itraconazole and/or vitamin D3.

2. The system of claim 1, wherein each microneedle has a diameter of less than 300 micrometers and a length of between 50 and 900 micrometers.

3. The system of claim 1, wherein the pharmacologic agent is disposed onto the surfaces of the microneedles via piezoelectric inkjet printing.

4. The system of claim 1, wherein the needles are lancet shaped.

5. The system of claim 1, wherein the distal ends of the microneedles deliver the pharmacologic agent to a skin site having basal cell carcinoma below a stratum corneum layer of the skin site.

6. The system of claim 1, wherein the microneedles are biodegradable.

7. The system of claim 1, wherein the microneedles are polyglycolic acid (PGA) polymer microneedles.

8. The system of claim 1, wherein the microneedles are metal.

9. The system of claim 1, wherein the array of microneedles comprises a circular array of microneedles.

10. The system of claim 8, wherein the array of microneedles occupies an area of 1 square centimeter.

11. The system of claim 1, wherein the array of microneedles comprises a rectangular array of microneedles.

12. The system of claim 1, wherein the microneedles are 800 micrometers long.

13. A method of treating basal cell carcinoma in a subject comprising:
applying an array of microneedles to a skin site having basal cell carcinoma, such that the microneedles penetrate the surface of the skin and the stratum corneum layer of the skin; and
administering a pharmaceutical composition to the subject intradermally through openings in the skin formed by the microneedles, wherein the pharmaceutical composition comprises a therapeutically effective amount of a pharmacologic chosen from itraconazole, vitamin D3, or a combination thereof.

14. The method of claim 13, wherein the pharmaceutical composition is applied intradermally after removing the microneedles from the skin.

15. The method of claim 13, wherein microneedles are hollow and define an opening at the distal end of each microneedle, and the pharmaceutical composition is applied intradermally through the openings of the microneedles.

16. The method of claim 13, wherein each microneedle has a diameter of less than 300 micrometers and a length of between 50 and 900 micrometers.

17. The method of claim 13, wherein the surfaces of the microneedles penetrate the surface of the skin and the stratum corneum layer of the skin.

18. The method of claim 13, wherein the pharmacologic composition is administered to the skin site having basal cell carcinoma until the basal cell carcinoma is no longer present.
